(19) United States
(12) Patent Application Publication
(10) Pub. No.: US 2022/0387696 A1
(43) Pub. Date: Dec. 8, 2022

(54) FLUID BRIDGE CONFIGURED FOR USE VERTICALLY AND/OR UNDER COMPRESSION

(71) Applicant: KCI Licensing, Inc., San Antonio, TX (US)

(72) Inventors: Justin Alexander LONG, Lago Vista, TX (US); Christopher Brian LOCKE, Bournemouth (GB)

(21) Appl. No.: 17/770,123

(22) PCT Filed: Oct. 15, 2020
(86) PCT No.: PCT/IB2020/059723

§ 371 Date: Apr. 19, 2022

Related U.S. Application Data
(60) Provisional application No. 62/924,012, filed on Oct. 21, 2019.

(51) Int. Cl. A61M 1/00

(52) U.S. Cl. CPC A61M 1/913; A61M 1/915; A61M 1/964; A61M 1/98

(57) ABSTRACT

Disclosed embodiments may relate to a fluid bridge configured to facilitate negative -pressure therapy when used in a vertical orientation and/or under compression, and to systems and methods related thereto. In some embodiments, the fluid bridge may comprise an open-cell foam support manifold within an envelope. The support manifold may be configured to reduce negative-pressure drop across the length of the fluid bridge and/or to maintain therapeutic levels of negative pressure, for example despite vertical orientation and/or use under a compression garment.

### CROSS-REFERENCE TO RELATED APPLICATIONS

This application claims the benefit of priority to U.S. Provisional Application No. 62/924,012, filed on Oct. 21, 2020, which is incorporated herein by reference in its entirety.

### TECHNICAL FIELD

The invention set forth in the appended claims relates generally to tissue treatment systems and more particularly, but without limitation, to low-profile distribution components for providing negative-pressure therapy.

### BACKGROUND

Clinical studies and practice have shown that reducing pressure in proximity to a tissue site can augment and accelerate growth of new tissue at the tissue site. The applications of this phenomenon are numerous, but it has proven particularly advantageous for treating wounds. Regardless of the etiology of a wound, whether trauma, surgery, or another cause, proper care of the wound is important to the outcome. Treatment of wounds or other tissue with reduced pressure may be commonly referred to as “negative-pressure therapy,” but is also known by other names, including “negative-pressure wound therapy,” “reduced-pressure therapy,” “vacuum therapy,” “vacuum-assisted closure,” and “topical negative-pressure,” for example. Negative-pressure therapy may provide a number of benefits, including migration of epithelial and subcutaneous tissues, improved blood flow, and micro-deformation of tissue at a wound site. Together, these benefits can increase development of granulation tissue and reduce healing times.

There is also widespread acceptance that cleansing a tissue site can be highly beneficial for new tissue growth. For example, a wound or a cavity can be washed out with a liquid solution for therapeutic purposes. These practices are commonly referred to as “irrigation” and “lavage”. “Instillation” is another practice that generally refers to a process of slowly introducing fluid to a tissue site and leaving the fluid for a prescribed period of time before removing the fluid. For example, instillation of topical treatment solutions over a wound bed can be combined with negative-pressure therapy to further promote wound healing by loosening soluble contaminants in a wound bed and removing infectious material. As a result, soluble bacterial burden can be decreased, contaminants removed, and the wound cleansed.

Additionally, a firm-fitting wrap or elastic bandage may be used to apply compression to a limb or other tissue site in some instances. Such compression therapy may be particularly beneficial for treating venous disease, such as leg ulcers or oedema. Compression of a leg, for example, may increase the pressure within veins of the leg and, particularly the veins proximate to a surface of the leg. Veins proximate to a surface of a leg may also be known as superficial veins. Compression may generally encourage blood flow from superficial veins toward deeper veins where blood may be more readily carried out of a leg. An increase in pressure in veins can decrease swelling and can reduce symptoms of venous leg disease, for example, promoting healing.

While the clinical benefits of negative-pressure therapy and/or instillation therapy and/or compression therapy are widely known, improvements to therapy systems, components, and processes may benefit healthcare providers and patients.

### BRIEF SUMMARY

New and useful systems, apparatuses, and methods for treating tissue in a negative-pressure therapy environment are set forth in the appended claims. Illustrative embodiments are also provided to enable a person skilled in the art to make and use the claimed subject matter.

For example, a negative pressure treatment system may use a fluid bridge to transfer negative pressure to a tissue site. A fluid bridge may be particularly helpful if the location of the tissue site makes application of negative pressure difficult and/or uncomfortable for a patient. Foot or leg wounds may be examples of such tissue sites, particularly if compression therapy may also be warranted. Thus, some examples of a fluid bridge may be configured to allow for negative-pressure therapy and compression therapy to be jointly applied in combination. In some applications, the fluid bridge may be used in an upright or vertical orientation. Portions of the fluid bridge may also be used under a compression garment on a leg or foot, and portions of the fluid bridge may extend upward out of the compression garment.

In more specific examples, the fluid bridge may be an apparatus for conducting fluid that may comprise a first barrier or envelope formed from a fluid-impermeable material, which defines a fluid path having two ends and a longitudinal axis. A manifolding material may be located within the envelope, serving as a support manifold that supports the envelope to define the fluid path. In some embodiments, the support manifold may comprise a dense open-cell foam material capable of resisting compressive effects. For example, the foam may be felted, by compression when heated. A felted foam may be characterized by a firmness factor, which is indicative of the compression of the foam. The firmness factor of a felted foam can be specified as the ratio of original thickness to final thickness. In some embodiments, the foam may be felted to a firmness factor of 2-5 or 3-5. In some embodiments, the foam may be open-cell polyurethane foam, such as found in GRANUFOAM™ Dressing, felted to a firmness factor of 3-5. In addition to increasing density of the foam, felting may also increase the number of tortuous pathways in a given thickness, which can help minimize negative-pressure drop across the length of the fluid bridge. Denser foam support manifold embodiments may allow the fluid bridge to operate better vertically and/or when used under a compression garment. For example, some fluid bridge embodiments may not suffer any substantial compression-related pressure drop and/or may better resist collapse.

More generally, some embodiments may relate to a fluid bridge for delivering negative-pressure to a tissue site, and may comprise a support manifold comprising open-cell foam having a thickness of at least 5 mm, a density of about 2.6-8.0 lb/f³, a free volume in a range of about 18% to about 45%, and/or about 80-250 pores per inch on average (e.g., as measured in the direction of compression); and an envelope encompassing the support manifold. The support manifold may support the envelope to define and/or maintain an enclosed fluid pathway within the envelope. In some embodiments, the envelope may comprise a first surface and a second surface; the bridge may comprise a first end and a second end; and the envelope may further comprise a first aperture located on the first surface in proximity to the first end and a second aperture located on the second surface in proximity to the second end. In some embodiments, the average pore size of the open cell foam of the support manifold may be about 80-300 micron (e.g., as measured in the direction of compression). The foam of the support manifold may be hydrophobic in some embodiments and/or may comprise polyurethane foam.

Fluid bridge embodiments may use a support manifold with a thickness of about 5-7 mm. In some embodiments, the foam of the support manifold may have a density of about 3.9-4.8 lb/ft³, the average pore size of the open-cell foam of the support manifold may be about 133-200 micron, the open-cell foam of the support manifold may have free volume in a range of about 18-30%, the open-cell foam of the support manifold may on average have 120-150 pores per inch, and/or the open-cell foam of the support manifold may have on average 120-135 pores per inch. The foam of some support manifold embodiments may have a 25% compression load deflection of at least 1.05 pounds per square inch and a 65% compression load deflection of at least 1.29 pounds per square inch, while other embodiments may have a 25% compression load deflection of at least 1.75 pounds per square inch and a 65% compression load deflection of at least 2.15 pounds per square inch. In some embodiments, the foam of the support manifold may comprise felted foam with a felted firmness factor of 2-5 or 3-5.

In some embodiments, the support manifold may be configured so that the negative-pressure drop across the length of the fluid bridge when in substantially vertical orientation is no more than about 45 mmHg. The support manifold of some embodiments may be configured so that the negative-pressure drop across the length of the fluid bridge when in substantially vertical orientation and under compression (e.g. under a compression garment) is no more than about 50 mmHg. Some embodiments of the fluid bridge may be configured to maintain, when used in a substantially vertical orientation and under compression, at least 75 mmHg negative pressure at a second aperture (e.g. located at the tissue site) when 125 mmHg negative pressure is applied to the first aperture.

Some embodiments may relate to a fluid bridge comprising: a support manifold comprising open-cell foam typically having a thickness of at least 5 mm, wherein the foam of the support manifold comprises felted foam with a felted firmness factor of 2-5; and an envelope encompassing the support manifold, wherein the support manifold supports the envelope. The foam of some support manifold embodiments may have a felted foam firmness factor of 3-5. In some embodiments, the foam of the support manifold may have pre-felted foam characteristics (e.g. of the foam blank to be felted to form the support manifold) of 40-50 pores per inch on average, pre-felted density of 1.3-1.6 lb/ft³, pre-felted average pore sizes of 400-600 micron, pre-felted free volume of about 90% or more, and/or pre-felted 25% compression load deflection of least 0.35 pounds per square inch and 65% compression load deflection of at least 0.43 pounds per square inch. In some embodiments, the foam blank may have a pre-felted thickness of 10-35 mm, 10-25 mm, 10-20 mm, or 15-20 mm. In some embodiments, the felted foam support manifold (e.g. after felting) may have a thickness of about 5-7 mm.

System embodiments for treating a tissue site with negative pressure may comprise: a fluid bridge having a support manifold; and a negative-pressure source in fluid communication with the support manifold at a first end of the fluid bridge; wherein the fluid bridge is oriented substantially vertically and a second end is in proximity to and in fluid communication with the tissue site. In some embodiments, a compression garment may be positioned to at least cover the second end of the fluid bridge. Typically, the compression garment may apply about 20-60 mmHg compression or about 40-60 mmHg compression, as needed for therapeutic compression effect. In some embodiments, the negative-pressure drop across the length of the bridge may be no more than about 50 mmHg, for example despite compression and vertical orientation. In some system embodiments, the bridge may maintain at least 75 mmHg negative pressure at the second end when 125 mmHg negative pressure is applied to the first end of the fluid bridge.

Some embodiments may relate to methods of applying negative pressure to a tissue site, and the methods may comprise the steps of: providing a fluid bridge having a support manifold with a first end and a second end; disposing the fluid bridge with the second end in proximity to the tissue site; providing a compression garment; disposing the compression garment to provide compression to the tissue site, wherein the second end is disposed under the compression garment; and applying negative pressure to the first end; wherein the second end maintains at least 75 mmHg negative pressure to the tissue site. In some embodiments, the pressure change between the first end and the second end of the fluid bridge may be no more than 50 mmHg. Disposing the fluid bridge may comprise orienting the fluid bridge substantially vertically, in some embodiments. Disposing the compression garment may comprise configuring the compression garment to apply about 20-60 mmHg compression or about 40-60 mmHg compression, in some embodiments. Typically, applying negative pressure may comprise applying from about 125 to about 150 mmHg of negative pressure to the first end, for example applying about 125 mmHg of negative pressure to the first end. In some embodiment, applying negative pressure may occur continuously for at least 30 hours, at least 40 hours, at least 50 hours, or at least 60 hours.

Some embodiments may relate to a method of forming a fluid bridge, and the method may comprise: providing an open-cell foam support manifold having a thickness of at least 5 mm, a density of about 2.6-8.0 lb/ft³, a free volume of about 18-45%, and/or about 80-250 pores per inch on average; and encasing the support manifold in an envelope, wherein the support manifold supports the envelope to define an enclosed fluid pathway. In some embodiments, the average pore size of the open-cell foam of the support manifold may be from 80-300 micron. In some embodiments, the foam of the support manifold may be hydrophobic. For example, the foam of the support manifold may comprises polyurethane foam. Typically, support manifold embodiments may have a thickness of about 5-7 mm. In some embodiments, the foam of the support manifold may have a density of about 3.9-4.8 lb/ft³, the average pore size of the open-cell foam of the support manifold may be from 133-200 micron, the open-cell foam of the support manifold may on average have 120-150 pores per inch, and/or the open-cell foam of the support manifold may on average have 120-135 pores per inch. In some embodiments, the envelope may comprise a first surface and a second surface; the fluid bridge may comprise a first end and a second end; and the method may further comprise forming a first aperture located on the first surface in proximity to the first end and a second aperture located on the second surface in proximity to the second end. Providing an open-cell foam support manifold may comprise: providing an open-cell foam blank having a thickness of at least 10 mm, 40-50 pores per inch on average, and a density of 1.3-1.6 lb/ft³, and felting the foam blank, in some embodiments. For example, the foam blank may be felted to 2-5 firmness or to 3-5 firmness. In some embodiments, the pre-felted foam blank may have pre-felted average pore sizes of 400-600 micron, a free volume of about 18-30%, and/or pre-felted thickness of 10-35 mm. Some embodiments may further comprise attaching a moisture-removal (e.g. wicking) layer adjacent to the second surface, opposite the support manifold. Some embodiments may further comprise forming a calibrated fluid flow in the envelope of less than about 5 cc/min located in proximity to the second end. In some embodiments, the method may further comprise: providing a hydrophilic foam layer; and disposing the hydrophilic foam layer in proximity to the support manifold within the envelope. In some embodiments, the first surface of the envelope may comprise an evaporative layer; and the hydrophilic layer may be disposed between the support manifold and the first surface.

Objectives, advantages, and a preferred mode of making and using the claimed subject matter may be understood best by reference to the accompanying drawings in conjunction with the following detailed description of illustrative embodiments.

### BRIEF DESCRIPTION OF THE DRAWINGS

FIG. 1 is a functional block diagram of an example embodiment of a therapy system that can provide negative-pressure treatment in accordance with this specification;

FIG. 2 is a schematic diagram of an example embodiment of the therapy system of FIG. 1 configured to apply negative pressure to a tissue site utilizing an exemplary fluid bridge;

FIG. 3A is a plan view of an exemplary fluid bridge;

FIG. 3B is a schematic, cross-sectional view of the fluid bridge of FIG. 3A taken along line 3B-3B;

FIG. 3C is an exploded view of the fluid bridge of FIG. 3A;

FIG. 4 is a schematic, cross-sectional view of another exemplary fluid bridge;

FIG. 5 is a chart of exemplary results illustrating performance of two vertically-oriented sample fluid bridges in comparison to a control sample; and

FIG. 6 is a chart of additional exemplary results illustrating performance of two vertically-oriented sample bridges in comparison to a control sample when used under a compression wrap.

### DESCRIPTION OF EXAMPLE EMBODIMENTS

The following description of example embodiments provides information that enables a person skilled in the art to make and use the subject matter set forth in the appended claims, but it may omit certain details already well known in the art. The following detailed description is, therefore, to be taken as illustrative and not limiting.

The example embodiments may also be described herein with reference to spatial relationships between various elements or to the spatial orientation of various elements depicted in the attached drawings. In general, such relationships or orientation assume a frame of reference consistent with or relative to a patient in a position to receive treatment. However, as should be recognized by those skilled in the art, this frame of reference is merely a descriptive expedient rather than a strict prescription.

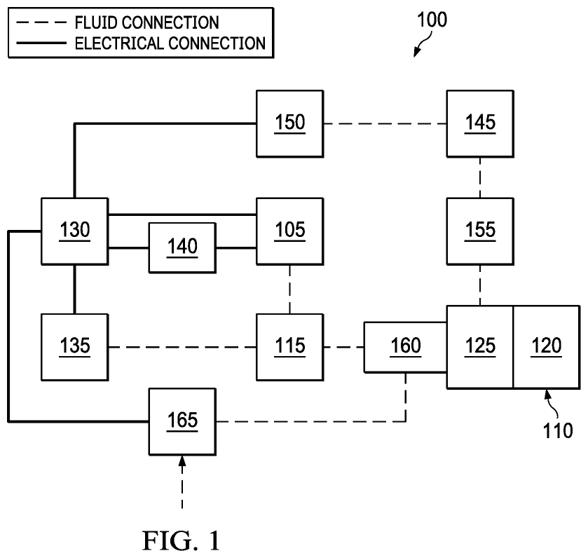

FIG. 1 is a simplified functional block diagram of an example embodiment of a therapy system 100 that can provide negative-pressure therapy with instillation of topical treatment solutions to a tissue site in accordance with this specification.

The term “tissue site” in this context broadly refers to a wound, defect, or other treatment target located on or within tissue, including, but not limited to, bone tissue, adipose tissue, muscle tissue, neural tissue, dermal tissue, vascular tissue, connective tissue, cartilage, tendons, or ligaments. A wound may include chronic, acute, traumatic, subacute, and dehisced wounds, partial-thickness burns, ulcers (such as diabetic, pressure, or venous insufficiency ulcers), flaps, and grafts, for example. The term “tissue site” may also refer to areas of any tissue that are not necessarily wounded or defective, but are instead areas in which it may be desirable to add or promote the growth of additional tissue. For example, negative pressure may be applied to a tissue site to grow additional tissue that may be harvested and transplanted.

The therapy system 100 may include a source or supply of negative pressure, such as a negative-pressure source 105, and one or more distribution components. A distribution component is preferably detachable and may be disposable, reusable, or recyclable. A dressing, such as a dressing 110, and a fluid container, such as a container 115, are examples of distribution components that may be associated with some examples of the therapy system 100. As illustrated in the example of FIG. 1, the dressing 110 may comprise or consist essentially of a tissue interface 120, a cover 125, or both in some embodiments.

A fluid conductor is another illustrative example of a distribution component. A “fluid conductor,” in this context, broadly includes a tube, pipe, hose, conduit, or other structure with one or more lumina or open pathways adapted to convey a fluid between two ends. A tube, for example, is generally an elongated, flexible structure with a cylindrical lumen, but the geometry and rigidity may vary. Moreover, some fluid conductors may be molded into or otherwise integrally combined with other components. Distribution components may also include or comprise interfaces or fluid ports to facilitate coupling and de-coupling other components. In some embodiments, for example, a dressing interface may facilitate coupling a fluid conductor to the dressing 110. For example, such a dressing interface may be a SENSAT.R.A.C.™ Pad, available from Kinetic Concepts, Inc. of San Antonio, Tex.

The therapy system 100 may also include a regulator or controller, such as a controller 130. Additionally, the therapy system 100 may include sensors to measure operating parameters and provide feedback signals to the controller 130 indicative of the operating parameters. As illustrated in FIG. 1, for example, the therapy system 100 may include a first sensor 135 and a second sensor 140 coupled to the controller 130.

In some embodiments, the therapy system 100 may also include a source of instillation solution. For example, a solution source 145 may be fluidly coupled to the dressing 110, as illustrated in the example embodiment of FIG. 1. The solution source 145 may be fluidly coupled to a positive-pressure source, such as a positive-pressure source 150, a negative-pressure source, such as the negative-pressure source 105, or both in some embodiments. A regulator, such as an instillation regulator 155, may also be fluidly coupled to the solution source 145 and the dressing 110 to ensure proper dosage of instillation solution (e.g. saline) to a tissue site. For example, the instillation regulator 155 may comprise a piston that can be pneumatically actuated by the negative-pressure source 105 to draw instillation solution from the solution source during a negative-pressure interval and to instill the solution to a dressing during a venting interval. Additionally or alternatively, the controller 130 may be coupled to the negative-pressure source 105, the positive-pressure source 150, or both, to control dosage of instillation solution to a tissue site. In some embodiments, the instillation regulator 155 may also be fluidly coupled to the negative-pressure source 105 through the dressing 110, as illustrated in the example of FIG. 1.

In some examples, a bridge 160 may fluidly couple the dressing 110 to the negative-pressure source 105, as illustrated in FIG. 1. The therapy system 100 may also comprise a flow regulator, such as a regulator 165, fluidly coupled to a source of ambient air to provide a controlled or managed flow of ambient air. In some embodiments, the regulator 165 may be fluidly coupled to the tissue interface 120 through the bridge 160. In some embodiments, the regulator 165 may be positioned proximate to the container 115 and/or proximate a source of ambient air, where the regulator 165 is less likely to be blocked during usage.

Some components of the therapy system 100 may be housed within or used in conjunction with other components, such as sensors, processing units, alarm indicators, memory, databases, software, display devices, or user interfaces that further facilitate therapy. For example, in some embodiments, the negative-pressure source 105 may be combined with the controller 130, the solution source 145, and other components into a therapy unit.

In general, components of the therapy system 100 may be coupled directly or indirectly. For example, the negative-pressure source 105 may be directly coupled to the container 115 and may be indirectly coupled to the dressing 110 through the container 115. Coupling may include fluid, mechanical, thermal, electrical, or chemical coupling (such as a chemical bond), or some combination of coupling in some contexts. For example, the negative-pressure source 105 may be electrically coupled to the controller 130 and may be fluidly coupled to one or more distribution components to provide a fluid path to a tissue site. In some embodiments, components may also be coupled by virtue of physical proximity, being integral to a single structure, or being formed from the same piece of material.

A negative-pressure supply, such as the negative-pressure source 105, may be a reservoir of air at a negative pressure or may be a manual or electrically-powered device, such as a vacuum pump, a suction pump, a wall suction port available at many healthcare facilities, or a micro-pump, for example. “Negative pressure” generally refers to a pressure less than a local ambient pressure, such as the ambient pressure in a local environment external to a sealed therapeutic environment. In many cases, the local ambient pressure may also be the atmospheric pressure at which a tissue site is located. Alternatively, the pressure may be less than a hydrostatic pressure associated with tissue at the tissue site. Unless otherwise indicated, values of pressure stated herein are gauge pressures. References to increases in negative pressure typically refer to a decrease in absolute pressure, while decreases in negative pressure typically refer to an increase in absolute pressure. While the amount and nature of negative pressure provided by the negative-pressure source 105 may vary according to therapeutic requirements, the pressure is generally a low vacuum, also commonly referred to as a rough vacuum, between −5 mmHg (−667 Pa) and −500 mmHg (−66.7 kPa). Common therapeutic ranges are between −50 mmHg (−6.7 kPa) and −300 mmHg (−39.9 kPa). For example, the therapeutic range applied to the tissue site may be at least −75 mmHg in some embodiments.

The container 115 is representative of a container, canister, pouch, or other storage component, which can be used to manage exudates and other fluids withdrawn from a tissue site. In many environments, a rigid container may be preferred or required for collecting, storing, and disposing of fluids. In other environments, fluids may be properly disposed of without rigid container storage, and a re-usable container could reduce waste and costs associated with negative-pressure therapy.

A controller, such as the controller 130, may be a microprocessor or computer programmed to operate one or more components of the therapy system 100, such as the negative-pressure source 105. In some embodiments, for example, the controller 130 may be a microcontroller, which generally comprises an integrated circuit containing a processor core and a memory programmed to directly or indirectly control one or more operating parameters of the therapy system 100. Operating parameters may include the power applied to the negative-pressure source 105, the pressure generated by the negative-pressure source 105, or the pressure distributed to the tissue interface 120, for example. The controller 130 is also preferably configured to receive one or more input signals, such as a feedback signal, and programmed to modify one or more operating parameters based on the input signals.

Sensors, such as the first sensor 135 and the second sensor 140, are generally known in the art as any apparatus operable to detect or measure a physical phenomenon or property, and generally provide a signal indicative of the phenomenon or property that is detected or measured. For example, the first sensor 135 and the second sensor 140 may be configured to measure one or more operating parameters of the therapy system 100. In some embodiments, the first sensor 135 may be a transducer configured to measure pressure in a pneumatic pathway and convert the measurement to a signal indicative of the pressure measured. In some embodiments, for example, the first sensor 135 may be a piezo-resistive strain gauge. The second sensor 140 may optionally measure operating parameters of the negative-pressure source 105, such as a voltage or current, in some embodiments. Preferably, the signals from the first sensor 135 and the second sensor 140 are suitable as an input signal to the controller 130, but some signal conditioning may be appropriate in some embodiments. For example, the signal may need to be filtered or amplified before it can be processed by the controller 130. Typically, the signal is an electrical signal, but may be represented in other forms, such as an optical signal.

The tissue interface 120 can be generally adapted to partially or fully contact a tissue site. The tissue interface 120 may take many forms, and may have many sizes, shapes, or thicknesses, depending on a variety of factors, such as the type of treatment being implemented or the nature and size of a tissue site. For example, the size and shape of the tissue interface 120 may be adapted to the contours of deep and irregular shaped tissue sites. Any or all of the surfaces of the tissue interface 120 may have an uneven, coarse, or jagged profile.

In some embodiments, the tissue interface 120 may comprise or consist essentially of a manifold. A manifold in this context may comprise or consist essentially of a means for collecting or distributing fluid across the tissue interface 120 under pressure. For example, a manifold may be adapted to receive negative pressure from a source and distribute negative pressure through multiple apertures across the tissue interface 120, which may have the effect of collecting fluid from across a tissue site and drawing the fluid toward the source. In some embodiments, the fluid path may be reversed or a secondary fluid path may be provided to facilitate delivering fluid, such as fluid from a source of instillation solution, across a tissue site.

In some illustrative embodiments, a manifold may comprise a plurality of pathways, which can be interconnected to improve distribution or collection of fluids. In some illustrative embodiments, a manifold may comprise or consist essentially of a porous material having interconnected fluid pathways. Examples of suitable porous material that can be adapted to form interconnected fluid pathways (e.g., channels) may include cellular foam, including open-cell foam such as reticulated foam; porous tissue collections; and other porous material such as gauze or felted mat that generally include pores, edges, and/or walls. Liquids, gels, and other foams may also include or be cured to include apertures and fluid pathways. In some embodiments, a manifold may additionally or alternatively comprise projections that form interconnected fluid pathways. For example, a manifold may be molded to provide surface projections that define interconnected fluid pathways.

In some embodiments, the tissue interface 120 may comprise or consist essentially of reticulated foam having pore sizes and free volume that may vary according to needs of a prescribed therapy. For example, reticulated foam having a free volume of at least 90% may be suitable for many therapy applications, and foam having an average pore size in a range of 400-600 microns (40-50 pores per inch) may be particularly suitable for some types of therapy. The tensile strength of the tissue interface 120 may also vary according to needs of a prescribed therapy. For example, the tensile strength of foam may be increased for instillation of topical treatment solutions. The 25% compression load deflection of the tissue interface 120 may be at least 0.35 pounds per square inch, and the 65% compression load deflection may be at least 0.43 pounds per square inch. In some embodiments, the tensile strength of the tissue interface 120 may be at least 10 pounds per square inch. The tissue interface 120 may have a tear strength of at least 2.5 pounds per inch. In some embodiments, the tissue interface may be foam comprised of polyols, such as polyester or polyether, isocyanate, such as toluene diisocyanate, and polymerization modifiers, such as amines and tin compounds. In some examples, the tissue interface 120 may be reticulated polyurethane foam such as found in GRANUFOAM™ dressing or V.A.C. VERAFLO™ dressing, both available from Kinetic Concepts, Inc. of San Antonio, Tex.

The thickness of the tissue interface 120 may also vary according to needs of a prescribed therapy. For example, the thickness of the tissue interface may be decreased to reduce tension on peripheral tissue. The thickness of the tissue interface 120 can also affect the conformability of the tissue interface 120. In some embodiments, a thickness in a range of about 5 millimeters to 10 millimeters may be suitable.

The tissue interface 120 may be either hydrophobic or hydrophilic. In an example in which the tissue interface 120 may be hydrophilic, the tissue interface 120 may also wick fluid away from a tissue site, while continuing to distribute negative pressure to the tissue site. The wicking properties of the tissue interface 120 may draw fluid away from a tissue site by capillary flow or other wicking mechanisms. An example of a hydrophilic material that may be suitable is a polyvinyl alcohol, open-cell foam such as V.A.C. WHITEFOAM™ dressing available from Kinetic Concepts, Inc. of San Antonio, Tex. Other hydrophilic foams may include those made from polyether. Other foams that may exhibit hydrophilic characteristics include hydrophobic foams that have been treated or coated to provide hydrophilicity.

In some embodiments, the tissue interface 120 may be constructed from bioresorbable materials. Suitable bioresorbable materials may include, without limitation, a polymeric blend of polylactic acid (PLA) and polyglycolic acid (PGA). The polymeric blend may also include, without limitation, polycarbonates, polyfumarates, and capralactones. The tissue interface 120 may further serve as a scaffold for new cell-growth, or a scaffold material may be used in conjunction with the tissue interface 120 to promote cell growth. A scaffold is generally a substance or structure used to enhance or promote the growth of cells or formation of tissue, such as a three-dimensional porous structure that provides a template for cell growth. Illustrative examples of scaffold materials include calcium phosphate, collagen, PLA/PGA, coral hydroxy apatites, carbonates, or processed allograft materials.

In some embodiments, the cover 125 may provide a bacterial barrier and protection from physical trauma. The cover 125 may also be constructed from a material that can reduce evaporative losses and provide a fluid seal between two components or two environments, such as between a therapeutic environment and a local external environment. The cover 125 may comprise or consist of, for example, an elastomeric film or membrane that can provide a seal adequate to maintain a negative pressure at a tissue site for a given negative-pressure source. The cover 125 may have a high moisture-vapor transmission rate (MVTR) in some applications. For example, the MVTR may be at least 250 grams per square meter per twenty-four hours in some embodiments, measured using an upright cup technique according to ASTM E96/E96M Upright Cup Method at 38° C. and 10% relative humidity (RH). In some embodiments, an MVTR up to 5,000 grams per square meter per twenty-four hours may provide effective breathability and mechanical properties.

In some example embodiments, the cover 125 may be a polymer drape, such as a polyurethane film, that is permeable to water vapor but impermeable to liquid. Such drapes typically have a thickness in the range of 25-50 microns. For permeable materials, the permeability generally should be low enough that a desired negative pressure may be maintained. The cover 125 may comprise, for example, one or more of the following materials: polyurethane (PU), such as hydrophilic polyurethane; cellulosics; hydrophilic polyamides; polyvinyl alcohol; polyvinyl pyrrolidone; hydrophilic acrylics; silicones, such as hydrophilic silicone elastomers; natural rubbers; polyisoprene; styrene butadiene rubber; chloroprene rubber; polybutadiene; nitrile rubber; butyl rubber; ethylene propylene rubber; ethylene propylene diene monomer; chlorosulfonated polyethylene; polysulfide rubber; ethylene vinyl acetate (EVA); co-polyester; and polyether block polymide copolymers. Such materials are commercially available as, for example, Tegaderm® drape, commercially available from 3M Company, Minneapolis Minn.; polyurethane (PU) drape, commercially available from Avery Dennison Corporation, Pasadena, Calif.; polyether block polyamide copolymer (PEBAX), for example, from Arkema S.A., Colombes, France; and Inspire 2301 and Inpsire 2327 polyurethane films, commercially available from Expopack Advanced Coatings, Wrexham, United Kingdom. In some embodiments, the cover 125 may comprise INSPIRE 2301 having an MVTR (upright cup technique) of 2600 g/m²/24 hours and a thickness of about 30 microns.

An attachment device may be used to attach the cover 125 to an attachment surface, such as undamaged epidermis, a gasket, or another cover. The attachment device may take many forms. For example, an attachment device may be a medically-acceptable, pressure-sensitive adhesive configured to bond the cover 125 to epidermis around a tissue site. In some embodiments, for example, some or all of the cover 125 may be coated with an adhesive, such as an acrylic adhesive, which may have a coating weight of about 25-65 grams per square meter (g.s.m.). Thicker adhesives, or combinations of adhesives, may be applied in some embodiments to improve the seal and reduce leaks. Other example embodiments of an attachment device may include a double-sided tape, paste, hydrocolloid, hydrogel, silicone gel, or organogel.

The solution source 145 may also be representative of a container, canister, pouch, bag, or other storage component, which can provide a solution for instillation therapy. Compositions of solutions may vary according to a prescribed therapy, but examples of solutions that may be suitable for some prescriptions include hypochlorite-based solutions, silver nitrate (0.5%), sulfur-based solutions, biguanides, cationic solutions, and isotonic solutions.

In operation, the tissue interface 120 may be placed within, over, on, or otherwise proximate to a tissue site. If the tissue site is a wound, for example, the tissue interface 120 may partially or completely fill the wound, or it may be placed over the wound. The cover 125 may be placed over the tissue interface 120 and sealed to an attachment surface near a tissue site. For example, the cover 125 may be sealed to undamaged epidermis peripheral to a tissue site. Thus, the dressing 110 can provide a sealed therapeutic environment proximate to a tissue site, substantially isolated from the external environment, and the negative-pressure source 105 can reduce pressure in the sealed therapeutic environment. In some embodiments, the regulator 165 may control the flow of ambient air to purge fluids and exudates from the sealed therapeutic environment.

The fluid mechanics of using a negative-pressure source to reduce pressure in another component or location, such as within a sealed therapeutic environment, can be mathematically complex. However, the basic principles of fluid mechanics applicable to negative-pressure therapy and instillation are generally well-known to those skilled in the art, and the process of reducing pressure may be described illustratively herein as “delivering,” “distributing,” or “generating” negative pressure, for example.

In general, exudate and other fluid flow toward lower pressure along a fluid path. Thus, the term “downstream” typically implies something in a fluid path relatively closer to a source of negative pressure or further away from a source of positive pressure. Conversely, the term “upstream” implies something relatively further away from a source of negative pressure or closer to a source of positive pressure. Similarly, it may be convenient to describe certain features in terms of fluid “inlet” or “outlet” in such a frame of reference. This orientation is generally presumed for purposes of describing various features and components herein. However, the fluid path may also be reversed in some applications, such as by substituting a positive-pressure source for a negative-pressure source, and this descriptive convention should not be construed as a limiting convention.

Negative pressure applied across the tissue site through the tissue interface 120 in the sealed therapeutic environment can induce macro-strain and micro-strain in the tissue site. Negative pressure can also remove exudate and other fluid from a tissue site, which can be collected in container 115.

In some embodiments, the controller 130 may receive and process data from one or more sensors, such as the first sensor 135. The controller 130 may also control the operation of one or more components of the therapy system 100 to manage the pressure delivered to the tissue interface 120. In some embodiments, controller 130 may include an input for receiving a desired target pressure and may be programmed for processing data relating to the setting and inputting of the target pressure to be applied to the tissue interface 120. In some example embodiments, the target pressure may be a fixed pressure value set by an operator as the target negative pressure desired for therapy at a tissue site and then provided as input to the controller 130. The target pressure may vary from tissue site to tissue site based on the type of tissue forming a tissue site, the type of injury or wound (if any), the medical condition of the patient, and the preference of the attending physician. After selecting a desired target pressure, the controller 130 can operate the negative-pressure source 105 in one or more control modes based on the target pressure and may receive feedback from one or more sensors to maintain the target pressure at the tissue interface 120.

In some embodiments, the controller 130 may have a continuous pressure mode, in which the negative-pressure source 105 is operated to provide a constant target negative pressure for the duration of treatment or until manually deactivated. Additionally or alternatively, the controller may have an intermittent pressure mode. For example, the controller 130 can operate the negative-pressure source 105 to cycle between a target pressure and atmospheric pressure. In some examples, the target pressure may be set at a value of 135 mmHg for a specified period of time (e.g., 5 min), followed by a specified period of time (e.g., 2 min) of deactivation. The cycle can be repeated by activating the negative-pressure source 105, which can form a square wave pattern between the target pressure and atmospheric pressure.

In some example embodiments, the increase in negative pressure from ambient pressure to the target pressure may not be instantaneous. For example, the negative-pressure source 105 and the dressing 110 may have an initial rise time, which can vary depending on the type of dressing and therapy equipment being used. For example, the initial rise time for one therapy system may be in a range of about 20-30 mmHg/second and in a range of about 5-10 mmHg/second for another therapy system. If the therapy system 100 is operating in an intermittent mode, the repeating rise time may be a value substantially equal to the initial rise time.

In other examples, a target pressure can vary with time in a dynamic pressure mode. For example, the target pressure may vary in the form of a triangular waveform, varying between a negative pressure of 50 and 135 mmHg with a rise time set at a rate of +25 mmHg/min. and a descent time set at −25 mmHg/min. In other embodiments of the therapy system 100, the triangular waveform may vary between negative pressure of 25 and 135 mmHg with a rise time set at a rate of +30 mmHg/min and a descent time set at −30 mmHg/min.

In some embodiments, the controller 130 may control or determine a variable target pressure in a dynamic pressure mode, and the variable target pressure may vary between a maximum and minimum pressure value that may be set as an input prescribed by an operator as the range of desired negative pressure. The variable target pressure may also be processed and controlled by the controller 130, which can vary the target pressure according to a predetermined waveform, such as a triangular waveform, a sine waveform, or a saw-tooth waveform. In some embodiments, the waveform may be set by an operator as the predetermined or time-varying negative pressure desired for therapy.

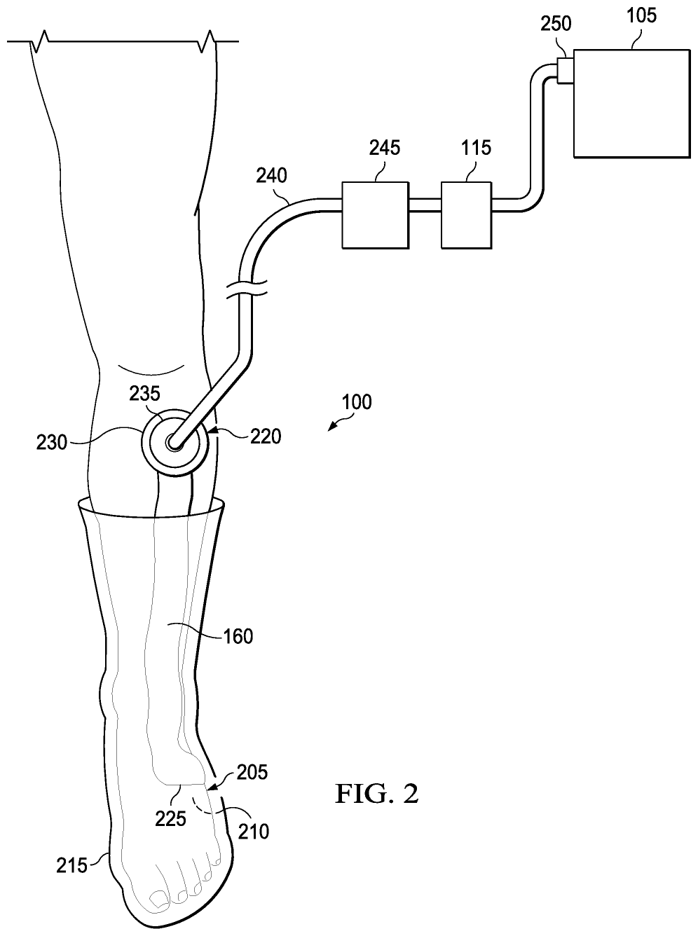

FIG. 2 is a schematic diagram of an example embodiment of the therapy system of FIG. 1 configured to apply negative pressure to a tissue site utilizing an exemplary fluid bridge 160, illustrating additional details that may be associated with some embodiments. The fluid bridge 160 may facilitate negative-pressure treatment of a tissue site 205, which in this illustration is on a patient's foot 210 and has limited access. In some embodiments, the treatment system 100 may be used with a tissue site having open access or limited access. An illustrative example of a limited-access tissue site may include a site under a compression garment 215.

The fluid bridge 160 may provide a low-profile path for negative pressure to be supplied to the tissue site 205 and thereby may increase patient comfort and enhance reliability of the negative-pressure supply to the tissue site 205. Because of the low profile of the fluid bridge 160, the fluid bridge 160 may readily be used with a compression garment 215. As such, the fluid bridge 160 may allow the patient the benefit of both negative-pressure treatment and compression treatment.

The fluid bridge 160 may have a first end 220 and a second end 225. The second end 225 may be placed proximate to the tissue site 205. In some embodiments, at least the second end 225 may be located under the compression garment 215. The first end 220 may typically be placed at a location on or near the patient that provides convenient access by the healthcare provider, such as a convenient location for applying negative pressure to the first end 220 of the fluid bridge 160. For example, first end 220 may be located away from the tissue site 205. In some embodiments, the first end 220 may be superior to the tissue site 205 (e.g. above the second end 225). In some embodiments, the first end 220 may be located external to the compression garment 215. As shown in FIG. 2, the fluid bridge 160 may be oriented substantially longitudinally (e.g. when the patient is ambulatory). For example, the first end 220 may be located at a vertical height greater than that of the second end 225. In some embodiments, the first end 220 may have a negative-pressure-interface site 230 that is configured for receiving a negative-pressure interface 235, which may be a port, such as SENSAT.R.A.C.™ technology from Kinetic Concepts, Inc. of San Antonio, Tex. If the compression garment 215 is utilized, the fluid bridge 160 may typically extend from the tissue site (e.g. under the compression garment 215) to a place outside of the compression garment 215. For example, the first end 220 of the fluid bridge 160 typically may be located outside the compression garment and/or at a location without substantial compression. The actual length of the fluid bridge 160 may be varied to support use with a particular compression garment 215 or application.

Compression may be applied to the tissue site 205 and/or to portions of the fluid bridge 160 in some treatment applications. For example, the compression garment 215 may be placed over at least a portion of the bridge 160 and/or the tissue site 205 in some embodiments. For example, the compression garment 215 may cover and/or apply compression to at least the second end 225 of the fluid bridge 160. In some embodiments, the compression garment 215 may apply 20-60 mmHg compression, 40-60 mmHg compression, or about 40 mmHg compression. Exemplary compression garments may include a compression wrap or bandage or a compression boot.

In some embodiments, a negative-pressure delivery conduit 240 may fluidly couple the negative-pressure interface 235 to a negative-pressure source 105. The negative-pressure source 105 may be any device or means for supplying a reduced pressure, such as a vacuum pump or wall suction. While the amount and nature of negative pressure applied to a site will vary according to the application, the negative pressure may typically be between 5 mmHg and 500 mmHg or more typically between 25 mmHg to 200 mmHg. For vertical applications of the fluid bridge 160, such as is shown in FIG. 2 on an ambulatory patient's leg, a specified minimum negative pressure may be necessary to ensure proper fluid flow. For example in some embodiments, a negative pressure of at least 125 mmHg may be a suitable minimum, but other pressures may be suitable for different situations. In some embodiments, the negative-pressure source 105 may provide about 125 mmHg negative pressure, or from 125 mmHg to 150 mmHg negative pressure may be provided. As used herein, “negative pressure” generally refers to a pressure less than the ambient pressure at a tissue site that is being subjected to treatment. In most cases, this negative pressure will be less than the atmospheric pressure at which the patient is located. Alternatively, the negative pressure may be less than a hydrostatic pressure at the tissue site. Unless otherwise indicated, values of pressure stated herein are gauge pressures. Although the terms “vacuum” and “negative pressure” may be used to describe the pressure applied to the tissue site, the actual pressure applied to the tissue site may be more than the pressure normally associated with a complete vacuum. Consistent with the use herein, an increase in negative pressure or vacuum pressure typically refers to a relative reduction in absolute pressure. In one illustrative embodiment, a V.A.C.® Therapy Unit by Kinetic Concepts, Inc. of San Antonio may be used as the negative-pressure source 105.

The therapy system 100 typically may be configured to maintain therapeutic levels of negative pressure to the tissue site 205, for example at least 75 mmHg negative pressure at the tissue site 205. For example, the fluid bridge 160 may be configured so that the negative-pressure drop across the length of the fluid bridge 160 in vertical orientation may be no more than 45 mmHg and/or so that the negative-pressure drop across the length of the fluid bridge 160 may be no more than 50 mmHg when the fluid bridge 160 is vertically oriented and under compression. In some embodiments, although the head pressure (e.g. due to vertical height across fluid bridge 160) may still be present, the configuration of the fluid bridge 160 may ensure that there is substantially no additional negative-pressure drop due to compression. For example, the negative-pressure drop across the length of the fluid bridge 160 attributable to compression (e.g. the compression garment 215) may be 5 mmHg or less after the pressure head due to height of vertically oriented fluid bridge 160 has built (e.g. after about 30 hours of negative-pressure therapy).

Depending on the application, a plurality of devices may be fluidly coupled to the negative-pressure delivery conduit 240. For example, a fluid container 115 or a device 245 may be included in some embodiments. The device 245 may be representative of another fluid reservoir or canister to hold exudates and other fluids removed in some embodiments. Other representative examples of the device 245 that may be included on the negative-pressure delivery conduit 240 may include the following non-limiting examples: a pressure-feedback device, a volume detection system, a blood detection system, an infection detection system, a flow monitoring system, a temperature monitoring system, a filter, etc. In some embodiments, some of these devices may be formed integral to the negative-pressure source 105. For example, a negative-pressure port 250 on the negative-pressure source 105 may include a filter member that includes one or more filters, e.g., an odor filter.

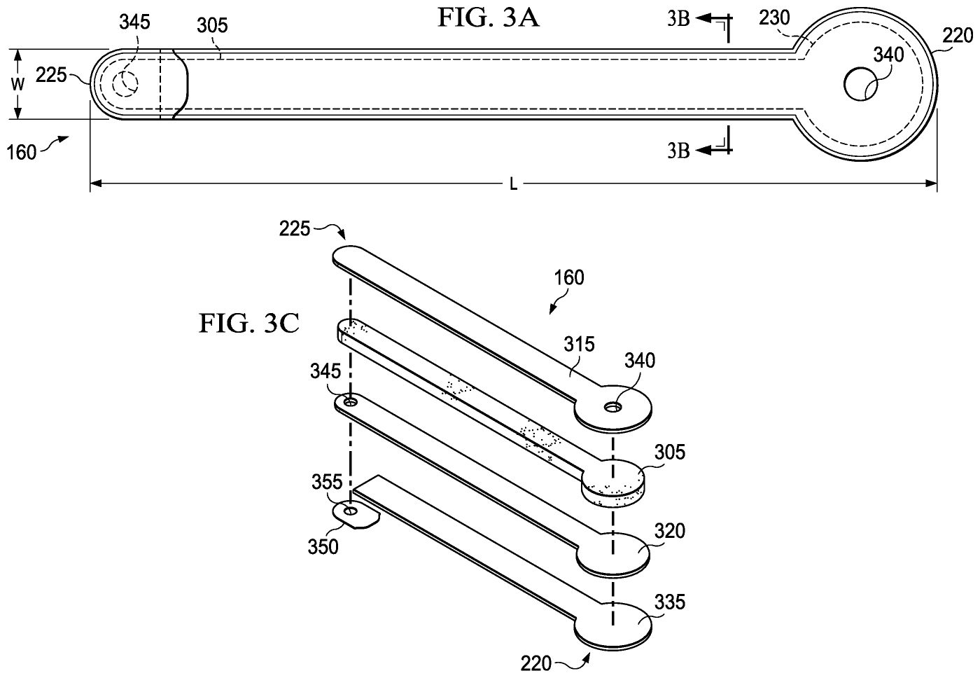

FIG. 3A is a plan view of another example of the fluid bridge 160, illustrating additional details that may be associated with some embodiments. The fluid bridge 160 may be elongated, having a length L and a width W. In some embodiments, the length L of the fluid bridge 160 may be substantially greater than the width W. The comfort or function of the fluid bridge 160 may be enhanced by using a length L to width W ratio that involves having the length dimension greater than the width. For example, in some embodiments, the relationship may be L>2W. In other illustrative embodiments, the relationship may be L>6W. In other illustrative embodiments, the relationship may be L>12W. In other illustrative embodiments, the relationship may be L>15W. In some illustrative embodiments, L may be approximately 668 mm and W may be approximately 56 mm. In some embodiments, the length L of the fluid bridge 160 may be about 650-670 mm, for example 650 mm. In some embodiments, the width of the fluid bridge 160 may be 40-56 mm. In some embodiments, the width of the fluid bridge 160 may be about 40 mm.

FIG. 3B is a schematic, cross-sectional view of the fluid bridge 160 of FIG. 3A taken along line 3B-3B, illustrating additional details that may be associated with some embodiments. As shown in FIG. 3B, the fluid bridge 160 may also have a thickness T. In some embodiments, the fluid bridge 160 may preferably have a low profile (e.g., a small dimension T), which may be as small as possible while providing adequate fluid flow for negative-pressure therapy. For non-limiting examples, T may be 30 mm, 20 mm, 15 mm, 10 mm, 5 mm, or less. For example, T may be greater than 5 millimeters, 5-10 millimeters, 5-7 millimeters, or 6-7 millimeters.

In some embodiments, the fluid bridge 160 may comprise a support manifold 305 encompassed by an envelope 310. The envelope 310 may define an internal fluid flow path or pathway from the first end 220 to the second end 225 (as shown in FIG. 3A). The envelope 310 may be made of a material that is impermeable to liquid and/or is substantially air-tight (e.g. allowing a vacuum to be drawn through the envelope). In some embodiments, the envelope 310 may comprise at least one vapor-transfer surface that is permeable to vapor. In some embodiments, the support manifold 305 may structurally support the envelope 310. For example, the support manifold 305 may be configured to maintain an open fluid flow path within the envelope 310 by maintaining space within the envelope 310 and/or preventing complete collapse of the envelope 310, for example due to negative pressure within the envelope 310 and/or application of compression forces on the fluid bridge 160. In some embodiments, the support manifold 305 may extend substantially the entire length L and/or width W and/or thickness T of the envelope 310, for example substantially filling the fluid flow path defined by the interior enclosed space of the envelope 310.

In some embodiments, the envelope 310 of FIG. 3B may comprise a first encapsulating member 315 and a second encapsulating member 320, for example with a periphery of the first encapsulating member 315 coupled to the second encapsulating member 320 to form the envelope 310 with fluid pathway between the two ends of the fluid bridge 160. In FIG. 3B, a periphery portion 325 of the first encapsulating member 315 and a periphery portion 330 of the second encapsulating member 320 may be coupled, such as by RF weld, to form the encapsulating envelope 310. The first encapsulating member 315 and the second encapsulating member 320 may be coupled using any technique, including without limitation welding (e.g., ultrasonic or RF welding), bonding, adhesives, cements, etc. The encapsulating envelope 310 may completely enclose the support manifold 305. Optionally, some embodiments may further comprise a moisture-removing device, such as wicking layer 335, which may be coupled to at least a portion of the encapsulating envelope 310 using any technique. For example, the wicking layer 335 may be coupled to an exterior side of the second encapsulating member 320 (e.g. opposite the support manifold 305).

FIG. 3C is an exploded view of the fluid bridge 160 of FIG. 3A, illustrating additional details associated with some embodiments. FIG. 3C illustrates exemplary layers that may make up the fluid bridge 160. For example, in FIG. 3C, the support manifold 305 may be located between the first encapsulating member 315 and the second encapsulating member 320. In some embodiments, the first encapsulating member 315 and the second encapsulating member 320 may each comprise a polymer film layer of liquid-impermeable material. The first encapsulating member 315 may be on a first side or surface of the fluid bridge 160, for example an outward-facing surface configured to face away from the patient. The first encapsulating member 315 may have a first aperture 340 formed proximate the first end 220. The first aperture 340 may allow fluid flow interaction between the internal flow path within the envelope 310 and an external negative-pressure source. Typically in use, the first aperture 340 may allow negative pressure application to the fluid bridge 160, for example with negative pressure entering the envelope 310 through the first aperture 340. The support manifold 305 may be disposed adjacent to the first encapsulating member 315. The second encapsulating member 320 may be disposed adjacent to the support manifold 305, opposite the first encapsulating member 315. For example, the second encapsulating member 320 may form the second, patient-facing side or surface of the fluid bridge 160. Thus, the support manifold 305 in FIG. 3C may be located between and in contact with the first encapsulating member 315 and the second encapsulating member 320. The second encapsulating member 320 may be formed with a second aperture 345 proximate the second end 225. In some embodiments, the second aperture 345 may allow fluid interaction between the internal flow path within the envelope 310 and an external environment. Typically in use, the second aperture 345 may allow exudate from the tissue site to enter the fluid bridge 160 under negative pressure.

In some embodiments, a moisture-removing device may be disposed adjacent to the second encapsulating member 320, opposite the support manifold 305 (e.g. coupled to a patient-facing side of the second encapsulating member 320). As illustrated in the example of FIG. 3C, the moisture-removing device may comprise a wicking layer 335 in some embodiments. In some embodiments, a releasable backing member or release liner 350 may be included on the second end 225 to releasably cover an adhesive. The releasable backing member 350 may be formed with an aperture 355 that aligns with the second aperture 345 in the second encapsulating member 320.

In some embodiments, the support manifold 305 may provide effective manifolding that distributes or collects fluid and/or negative pressure within the fluid bridge. For example, the support manifold 305 may receive negative pressure from a source at the first end 220 (e.g. at the first aperture 340) and distribute negative pressure through multiple apertures in the fluid bridge 160, which may have the effect of drawing fluid from the second end 225 towards the negative pressure source. The support manifold 305 may comprise any material capable of transferring negative pressure. In some embodiments, the support manifold 305 may be formed from the same material as the tissue interface 120. The support manifold 305 may have any thickness but typically may have a low profile, such as a thickness in the range of 3-20 millimeters, 5-10 millimeters, 5-7 millimeters, 6-7 millimeters, etc. In some embodiments, the support manifold 305 may be at least 5 millimeters thick. The thickness of the support manifold 305 may be varied to minimize or eliminate pressure points on the tissue site. The thickness of the support manifold 305 may also be selected to support fluid removal from the tissue site and transfer into a canister, such as the fluid canister 115 in FIG. 2.

In some embodiments, the support manifold 305 may comprise an open-cell foam having a free volume in a range of about 18% to about 45%, a density of about 2.6-8.0 lb/ft³, about 80-250 pores per inch on average (e.g., as measured in the direction of compression), and/or average pore size of about 80-300 micron (e.g., as measured in the direction of compression), which may be particularly advantageous in a vertical orientation and/or under compression. For example, the denser foam may better resist the compressive effects when used under a compression garment and/or may better maintain fluid flow when under compression. In some embodiments, the density of the foam of the support manifold 305 may be about 3.9-4.8 lb/ft³. In some embodiments, the free volume of the foam may be in a range of about 18% to about 30%. In some embodiments, the free volume of the foam may be about 30%. In some embodiments, the average pore size of the support manifold 305 may be about 133-200 micron. In some embodiments, the support manifold 305 may have 120-150 pores per inch on average. For example, the support manifold 305 may comprise or consist essentially of a foam having 120-135 pores per inch on average. In some embodiments, the support manifold may have a 25% compression load deflection of at least 1.05 pounds per square inch and a 65% compression load deflection of at least 1.29 pounds per square inch. In some embodiments, the foam of the support manifold 305 may have a 25% compression load deflection of at least 1.75 pounds per square inch and a 65% compression load deflection of at least 2.15 pounds per square inch. In some embodiments, the foam of the support manifold 305 may have a 25% compression load deflection of about 1.05-1.75 pounds per square inch and a 65% compression load deflection of about 1.29-2.15 pounds per square inch.

In some embodiments, the support manifold 305 may be configured to maintain a thickness greater than 0.75 mm, 1 mm, or 1.25 mm when vertically oriented, under compression wrap, and/or experiencing at least 125 mmHg negative pressure. In some embodiments, the support manifold 305 may be configured so that the negative-pressure drop across the length of the fluid bridge 160 due to compression (e.g. from a compression garment) may be 5 mmHg or less after the fluid bridge 160 has been in continuous use for 30 hours or more. In some embodiments, the support manifold 305 may be configured so that substantially any pressure drop may be due to the pressure head of the vertical column of exudate (e.g. without any substantial contribution from compression). In some embodiments, the fluid bridge 160 configuration may ensure that the width W of the fluid bridge 160 may decrease less than 1 mm (e.g. about 0.8 mm) or less than 2 mm (e.g. about 1.9 mm) upon application of negative pressure (e.g. depending on the felting factor of the foam of the support manifold 305). In some embodiments, the fluid bridge 160 configuration may ensure that the width W of the fluid bridge 160 may decrease less than 10%, less than 5%, or about 2% or less upon application of negative pressure (e.g. 125 mmHg of negative pressure).

In some embodiments, the support manifold 305 may be formed by a felting process. Any porous foam suitable for felting may be used, including the example foams mentioned herein, such as found in GRANUFOAM™ dressing. In general, felting comprises a thermoforming process that permanently compresses a foam to increase the density of the foam while maintaining interconnected pathways. For example, in order to create felted foam, such as felted polyurethane, a foam blank of uncompressed material can be heated to an optimum forming temperature and then compressed. The felting process may alter certain properties of the original material, including pore shape and/or size, elasticity, density, and density distribution. For example, struts that define pores in the foam may be deformed during the felting process, resulting in flattened pore shapes. The deformed struts can also decrease the elasticity of the foam. The density of the foam is generally increased by felting. In some embodiments, contact with hot-press platens in the felting process can also result in a density gradient in which the density is greater at the surface and the pores size is smaller at the surface.

A felted foam may be characterized by a firmness factor, which is indicative of the compression of the foam. The firmness factor of a felted foam can be specified as the ratio of original thickness to final thickness. A compressed or felted foam may have a firmness factor greater than 1. The degree of compression may affect the physical properties of the felted foam. For example, felted foam has an increased effective density compared to a foam of the same material that is not felted. The felting process can also affect fluid-to-foam interactions. For example, as the density increases, compressibility or collapse may decrease. Therefore, foams which have different compressibility or collapse may have different firmness factors. In some example embodiments, a support manifold firmness factor can range from about 2 to about 5, preferably about 3 to about 5. For example, the firmness factor of the support manifold felted foam may be about 3 in some embodiments. There is a general linear relationship between firmness level, density, pore size (or pores per inch) and compressibility. For example, foam found in a GRANUFOAM™ dressing that is felted to a firmness factor of 3 will show a three-fold density increase and compress to about a third of its original thickness.

In some embodiments, a suitable foam blank (e.g. of pre-felted foam) for formation of the support manifold 305 may have 40-50 pores per inch on average, a density of 1.3-1.6 lb/ft³, a free volume of about 90% or more, an average pore size in a range of 400-600 micron, a 25% compression load deflection of at least 0.35 pounds per square inch, and/or a 65% compression load deflection of at least 0.43 pounds per square inch. In some embodiments, the foam blank may have a thickness greater than 10 mm, for example 10-35 mm, 10-25 mm, 10-20 mm, or 15-20 mm. In some embodiments, the foam blank may be felted to provide denser foam for the support manifold 305. For example, the foam blank may be felted to a firmness factor of 2-5. In some embodiments, the foam blank may be felted to a firmness factor of 3-5. Some embodiments may felt the foam blank to a firmness factor of 3.

Based for example on the foam selected for the fluid bridge 160 and/or the amount of felting, some fluid bridge 160 embodiments may be configured to reduce pressure drop across the length of the fluid bridge 160 (e.g. between the first end 220 and the second end 225 or the first aperture 340 and the second aperture 345) when the fluid bridge 160 is oriented substantially vertically and/or is under compression (e.g. due to application of a compression garment over at least a portion of the fluid bridge 160). For example, the negative-pressure drop across the length of the fluid bridge 160 (e.g. between the first end 220, where negative pressure is applied from the negative-pressure source 105, and the second end 225, where negative pressure is applied to the tissue site) may be no more than about 45 mmHg (e.g. if vertically oriented) or no more than about 50 mmHg (e.g. if vertically oriented and under compression). In some embodiments, the negative-pressure drop across the vertical length of the fluid bridge 160 may be less than 60 mmHg (e.g. when substantially vertically oriented) or less than 75 mmHg (e.g. when substantially vertically oriented and under compression). In some embodiments, the fluid bridge 160 may be configured to maintain at least 75 mmHg negative pressure at the second end 225 when 125 mmHg negative pressure is applied at the first end 220, even when used vertically and/or under compression.

The first encapsulating member 315 and the second encapsulating member 320 may be composed of any material that facilitates maintaining negative pressure within the encapsulating envelope formed from the first encapsulating member 315 and the second encapsulating member 320. In some embodiments, the first encapsulating member 315 and the second encapsulating member 320 may be formed of the same material as the cover 125. In some embodiments, the first encapsulating member 315 and the second encapsulating member 320 may comprise or consist essentially of a polyurethane film, but any suitable drape material may be readily used, such as any natural rubbers, polyisoprene, styrene butadiene rubber, chloroprene rubber, polybutadiene, nitrile rubber, butyl rubber, ethylene propylene rubber, ethylene propylene diene monomer, chlorosulfonated polyethylene, polysulfide rubber, polyurethane, EVA film, co-polyester, silicones, 3M Tegaderm® drape material, or acrylic drape material, such as one available from Avery. In some embodiments, the polyurethane film may have a thickness of 10-100 micron. For example, the film may be 0.75-1.5 Mil or about 30 micron thick polyurethane. In some embodiments, at least one of the films may be hydrophilic. In some embodiments, the first encapsulating member 315 and the second encapsulating member 320 may not both comprise the same material and/or have the same thickness. Some embodiments may comprise a perforation (not shown) forming a calibrated flow into the envelope, for example located in proximity to the second end 225 of the fluid bridge 160. For example, the calibrated flow may be about 5 cc/min or less in some embodiments.

The wicking layer 335 or other moisture-removing device may be configured to pull moisture, e.g., perspiration, away from a patient's skin and may help to substantially reduce or prevent maceration of the patient's skin and enhance comfort. The extent of the moisture-removing device can be varied both laterally (width) and longitudinally (lengthwise). For example, the moisture-removing device may cover 100 percent or more than 90 percent, 80 percent, 70 percent, 60 percent, or 50 percent of the patient-facing second encapsulating member 315. The wicking layer 335 or moisture-removing device may be configured to pull moisture to a place where the moisture can evaporate more readily. In some embodiments, the wicking layer 335 may be a film, cloth-material drape, a non-woven fabric, a knitted polyester woven textile material, such as the one sold under the name InterDry® AG material from Coloplast A/S of Denmark, GORTEX® material, DuPont Softesse® material, etc.

In some embodiments, apertures (not shown) may be formed on the second encapsulating member 320 that allow the negative pressure in the encapsulating envelope 310 to pull fluids from the wicking layer 335 into the support manifold 305. For example, apertures may be formed in the second encapsulating member 320 that allow the negative pressure within the encapsulating envelope 310 to pull fluids into the support manifold 305. In some embodiments, the apertures may comprise reduced-pressure valves configured to close in the absence of reduced pressure.

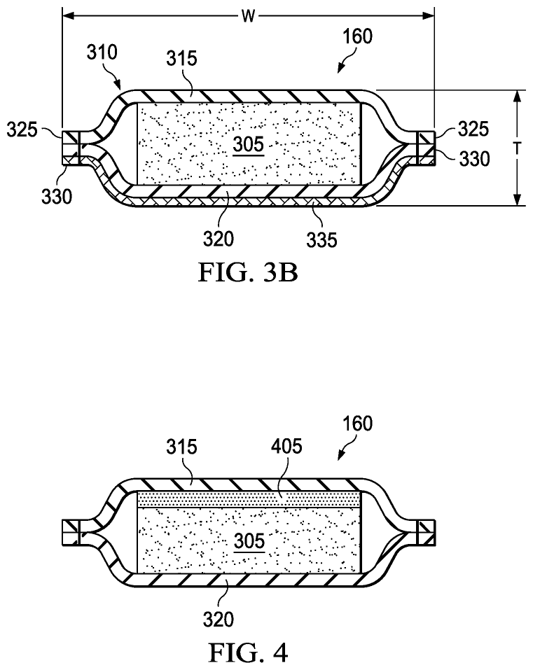

FIG. 4 is a cross-section view of another example of a fluid bridge 160, illustrating additional details that may be associated with some embodiments. The fluid bridge 160 of FIG. 4 may be similar to that of FIG. 3B, but may also comprise a hydrophilic layer 405 located within the envelope. For example, the hydrophilic layer 405 may be oriented to face away from the patient when the fluid bridge 160 is applied to the tissue site, with the support manifold 305 between the hydrophilic layer 405 and the patient. In FIG. 4, the hydrophilic layer 405 may be located between and in contact with the support manifold 305 and the first encapsulating member 315, while the support manifold 305 may be located between and in contact with the hydrophilic layer 405 and the second encapsulating member 320. The hydrophilic layer 405 in some embodiments may comprise hydrophilic open-cell foam configured to wick fluid away from the support manifold 305. In some embodiments, the outer surface of the envelope (e.g. the first encapsulating member 315) may comprise an evaporative material and/or be configured with a sufficiently thin thickness and/or a sufficiently vapor-permeable material to allow evaporation of some fluid within the envelope.

Some embodiments may be directed to a method of treating a tissue site with negative pressure. For example, methods of applying negative pressure to a tissue site may comprise the steps of: providing a fluid bridge having a support manifold with a first end and a second end; disposing the fluid bridge with the second end in proximity to the tissue site; providing a compression garment; disposing the compression garment to provide compression to the tissue site, wherein the second end is disposed under the compression garment; and applying negative pressure to the first end. In some embodiments, the second end may maintain at least −75 mmHg to the tissue site (e.g. 75 mmHg of negative-pressure). In some embodiments, the negative-pressure drop (e.g. change) between the first end and the second end may be no more than 50 mmHg. The step of disposing or placing the fluid bridge may comprise orienting the fluid bridge substantially vertically, in some embodiments. The step of disposing or placing the compression garment may comprise configuring the compression garment to apply about 20-60 mmHg or about 40-60 mmHg compression, in some embodiments. The step of applying negative pressure may in some embodiments comprise applying negative pressure of about 125 mmHg, at least 125 mmHg, or from about 125 to about 150 mmHg to the first end. In some embodiments, applying negative pressure may occur continuously for at least 60 hours (e.g. without negative-pressure drop of more than 50 mmHg and/or with at least 75 mmHg negative pressure being available at the second end). In some embodiments, the width of the fluid bridge may decease less than 1 mm (e.g. about 0.8 mm) or less than 2 mm (e.g. about 1.8 mm) upon application of negative pressure (depending on the felting firmness factor of the support manifold). In some embodiments, the support manifold may comprise open-cell foam, for example as set forth in the example fluid bridge embodiments described above.

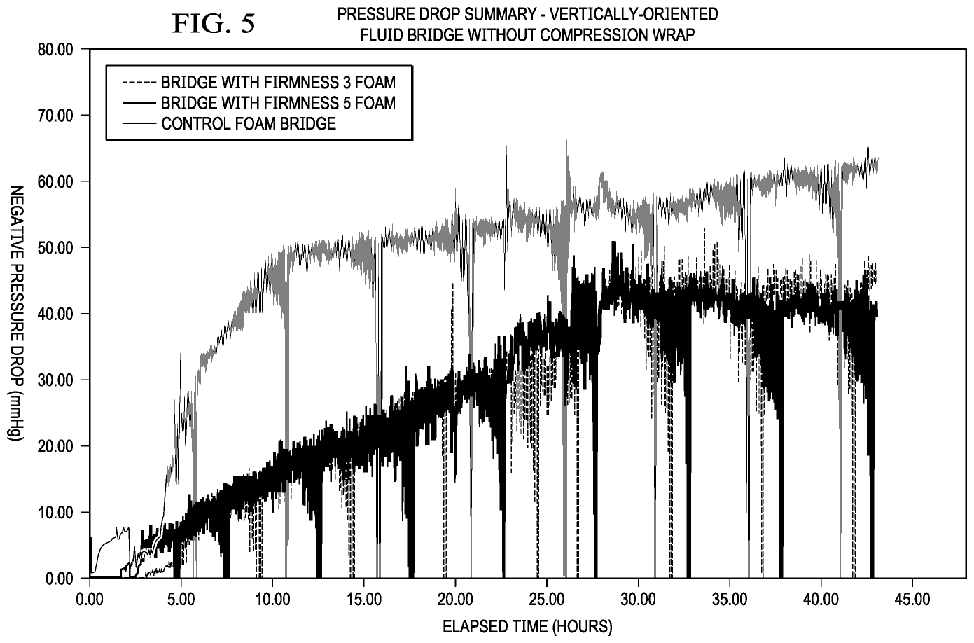

FIG. 5 is a chart illustrating the effectiveness of exemplary fluid bridges having a felted foam versus a control bridge having un-felted foam in use for negative-pressure therapy. In FIG. 5, the only difference between the three bridges tested was the firmness factor of the foam support manifold. One felted foam sample had a firmness factor of 3, and the other had a firmness factor of 5. The control bridge had a firmness factor of 1. All three bridges received 125 mmHg negative pressure. The fluid bridges in this example were all oriented vertically, and were not used under a compression wrap. As FIG. 5 shows, both felted bridges produced significantly less negative-pressure drop across the length of the bridge (e.g. an improvement of about 10 mmHg or better). The negative-pressure drop for the control bridge eventually passed 50 mmHg and then 60 mmHg in the chart, which means that the therapeutic minimum goal of 75 mmHg negative pressure was no longer being supplied to the tissue site. The felted bridges allowed more of the provided negative-pressure from the source to be applied to the tissue site, and ensured that at least 75 mmHg negative pressure was applied to the tissue site.

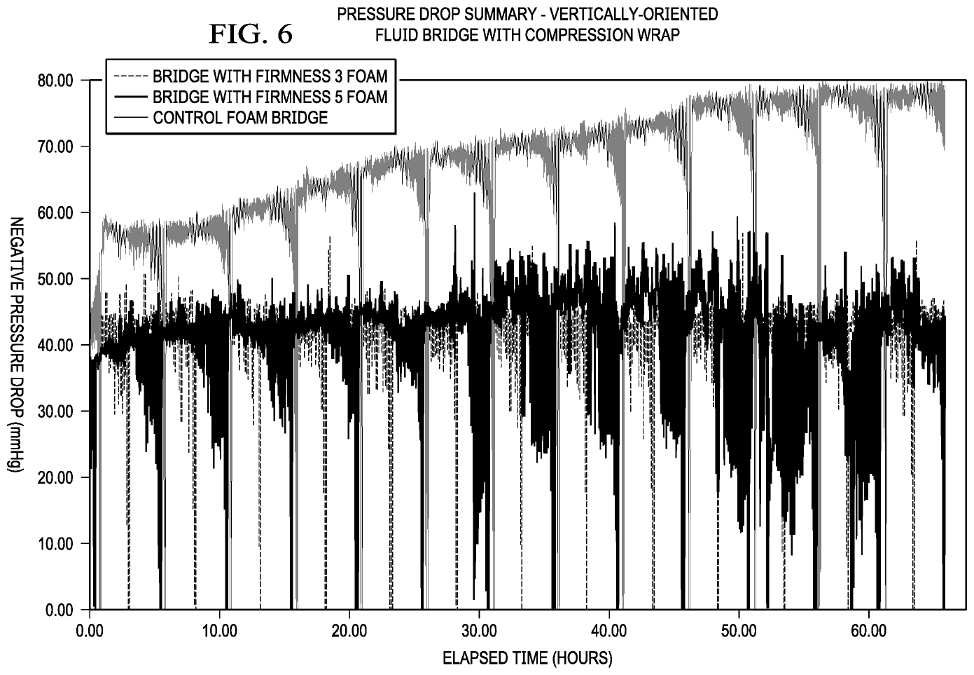

FIG. 6 similarly illustrates in a chart the effectiveness of exemplary felted foam fluid bridges versus a control bridge with un-felted foam, when used for negative-pressure therapy. In FIG. 6, the comparison occurs while the bridges are used with a compression wrap (e.g. with about 40 mmHg compression applied to the tissue site) and vertically oriented. Again, both felted bridges produced significantly less negative-pressure drop across the length of the bridge (e.g. an improvement of about 20 mmHg or better over the control bridge results). The negative-pressure drop for the un-felted bridge quickly passed 50 and 60 mmHg in the chart to approach 80 mmHg, which means that the therapeutic minimum goal of 75 mmHg negative pressure was no longer being supplied to the tissue site. The felted bridges allowed more of the provided negative-pressure from the source to be applied to the tissue site, and ensured that at least 75 mmHg negative pressure was applied to the tissue site. It may also be noted by comparing FIGS. 5 and 6 that, after the head pressure had time to build (e.g. about 30 hours), there was substantially no additional negative-pressure drop caused by the addition of compression. This may demonstrate the effectiveness of the felted foam fluid bridges when the negative-pressure therapy occurs in parallel to compressive therapy.

Some embodiments may be directed to a method of forming a fluid bridge, for example a fluid bridge configured for use vertically and/or under compression. For example, methods of forming a fluid bridge may comprise the steps of: selecting and/or providing an open-cell foam support manifold having a thickness of at least 5 mm, a density of about 2.6-8.0 lb/ft3, a free volume of about 18-45% (e.g. 18-30%), and/or about 80-250 pores per inch on average; and encasing the support manifold in an envelope, wherein the support manifold supports the envelope to define an enclosed fluid pathway. In some embodiments, the average pore size of the open-cell foam of the support manifold may range from 80-300 micron. In some embodiments, the foam selected for the support manifold may be hydrophobic. The foam of some support manifold embodiments may comprise polyurethane foam. In some embodiments, the support manifold foam may have a thickness of about 5-7 mm, may have a density of about 3.9-4.8 lb/ft3, may have average pore size of 133-200 micron, may on average have 120-150 pores per inch, may on average have 120-135 pores per inch, may have a 25% compression load deflection of at least 1.05 pounds per square inch and a 65% compression load deflection of at least 1.29 pounds per square inch, may have a 25% compression load deflection of at least 1.75 pounds per square inch and a 65% compression load deflection of at least 2.15 pounds per square inch, and/or may have a 25% compression load deflection of about 1.05-1.75 pounds per square inch and a 65% compression load deflection of about 1.29-2.15 pounds per square inch. In some embodiments, the foam of the support manifold may be selected to ensure that the fluid bridge maintains at least −75 mmHg at the second aperture when −125 mmHg is applied to the first aperture and/or that the negative-pressure drop across the length of the fluid bridge is no more than about 45 mmHg or about 50 mmHg (e.g. when the fluid bridge is oriented substantially vertically and/or is used under compression).

In some embodiments, the envelope may comprise a first surface and a second surface; the fluid bridge may comprise a first end and a second end; and the method may further comprise forming a first aperture located on the first surface in proximity to the first end and a second aperture located on the second surface in proximity to the second end. Some method embodiments may further comprise forming the open-cell foam support manifold, which may comprise: providing an open-cell foam blank (e.g. pre-felted foam) having thickness of at least 10 mm, 40-50 pores per inch on average, a free volume of about 90% or more, and/or density of 1.3-1.6 lb/ft3; and felting the foam blank. In some embodiments, felting the foam blank may comprise felting the foam blank to 2-5 firmness or to 3-5 firmness (for example, felted firmness factor 3). In some embodiments, the pre-felted foam (e.g. foam blank) may have average pore size of 400-600 micron; the pre-felted foam may have thickness of 10-35 mm; and/or the pre-felted foam may have 25% compression load deflection of at least 0.35 pounds per square inch, and 65% compression load deflection of at least 0.43 pounds per square inch.

Some method embodiments may further comprise attaching a moisture-removal layer adjacent to the second surface, opposite the support manifold. Some embodiments may further comprise forming a calibrated leak in the envelope of less than about 5 cc/min located in proximity to the second end. Some method embodiments may further comprise: providing a hydrophilic foam layer; and disposing or placing the hydrophilic foam layer in proximity to the support manifold within the envelope. The first surface of the envelope may comprise an evaporative layer in some embodiments, and the hydrophilic layer may be disposed between the support manifold and the first surface.

The systems, apparatuses, and methods described herein may provide significant advantages. For example, some embodiments may provide a means for applying therapeutic negative pressure to a tissue site, and may be particularly effective if used in a vertical orientation and/or under compression. Accordingly, the patient may benefit from both compressive therapy and negative-pressure therapy in some applications. For example, simultaneous negative-pressure therapy and compressive therapy may be helpful in treating diabetic foot ulcer or venous leg ulcer. The configuration of the fluid bridge embodiments may allow for effective therapeutic negative-pressure therapy when the tissue site is located under a compression garment, resisting the compressive effects and maintaining sufficient open fluid flow pathways to maintain required negative pressure at the tissue site. The configuration of the fluid bridge embodiments may also allow for effective negative-pressure therapy at the tissue site despite the head pressure from vertical orientation. Pressure drop across the vertical length of the fluid bridge may be minimized, for example preventing severe pressure and fluid manifolding drops that could negatively impact negative-pressure therapy. In some embodiments, the pressure drop across the fluid bridge may be substantially similar (e.g. within about 5 mmHg) in a vertical orientation regardless of whether the fluid bridge is used under a compression garment or not. Some embodiments may substantially reduce or eliminate additional pressure drop induced by a compression garment. Some embodiments may minimize skin maceration issues.

While shown in a few illustrative embodiments, a person having ordinary skill in the art will recognize that the systems, apparatuses, and methods described herein are susceptible to various changes and modifications that fall within the scope of the appended claims. For example, the fluid bridge 160 may be used in other settings, for example without a compression garment and/or linking multiple tissue sites. In some embodiments, the fluid bridge 160 may be used for one or more tissue sites which may result in some or all of the patient's body weight compressing the fluid bridge.

Moreover, descriptions of various alternatives using terms such as “or” do not require mutual exclusivity unless clearly required by the context, and the indefinite articles “a” or “an” do not limit the subject to a single instance unless clearly required by the context. Components may be also be combined or eliminated in various configurations for purposes of sale, manufacture, assembly, or use. For example, in some configurations the dressing 110, the container 115, or both may be separated from other components for manufacture or sale. In other example configurations, the controller 130 may also be manufactured, configured, assembled, or sold independently of other components.

The appended claims set forth novel and inventive aspects of the subject matter described above, but the claims may also encompass additional subject matter not specifically recited in detail. For example, certain features, elements, or aspects may be omitted from the claims if not necessary to distinguish the novel and inventive features from what is already known to a person having ordinary skill in the art. Features, elements, and aspects described in the context of some embodiments may also be omitted, combined, or replaced by alternative features serving the same, equivalent, or similar purpose without departing from the scope of the invention defined by the appended claims.

What is claimed is:

1. A fluid bridge for delivering negative-pressure to a tissue site, comprising:
a support manifold comprising open-cell foam having a density in a range of about 2.6-8.0 lb/ft³ and a free volume in a range of about 18% to about 45%; and
an envelope encompassing the support manifold, wherein the support manifold supports the envelope to maintain a fluid pathway within the envelope.

2. The fluid bridge of claim 1, wherein:
the envelope comprises a first surface and a second surface;
the support manifold comprises a first end and a second end; and
the envelope further comprises a first aperture located on the first surface in proximity to the first end and a second aperture located on the second surface in proximity to the second end.

3. The fluid bridge of any of claims 1-2, wherein the support manifold comprises a thickness of at least 5 mm.

4. The fluid bridge of any of claims 1-3, wherein the open-cell foam has about 80-250 pores per inch on average.

5. The fluid bridge of any of claims 1-4 wherein the open-cell foam has an average pore size of about 80-300 micron.

6. The fluid bridge of any of claims 1-5, wherein the open-cell foam of the support manifold is hydrophobic.

7. The fluid bridge of any of claims 1-6, wherein the open-cell foam of the support manifold comprises polyurethane foam.

8. The fluid bridge of any of claims 1-7, wherein the support manifold has a thickness of about 5-7 mm.

9. The fluid bridge of any of claims 1-8, wherein the open-cell foam of the support manifold has a density of about 3.9-4.8 lb/ft³.

10. The fluid bridge of any of claims 1-9, wherein the open-cell foam of the support manifold has an average pore size of about 133-200 micron.

11. The fluid bridge of any of claims 1-10, wherein the open-cell foam of the support manifold has 120-150 pores per inch on average.

12. The fluid bridge of any of claims 1-10, wherein the open-cell foam of the support manifold has 120-135 pores per inch on average.

13. The fluid bridge of any of claims 1-12, wherein the open-cell foam of the support manifold has a 25% compression load deflection of at least 1.05 pounds per square inch and a 65% compression load deflection of at least 1.29 pounds per square inch.

14. The fluid bridge of any of claims 1-12, wherein the open-cell foam of the support manifold has a 25% compression load deflection of at least 1.75 pounds per square inch and a 65% compression load deflection of at least 2.15 pounds per square inch.

15. The fluid bridge of any of claims 1-12, wherein the open-cell foam of the support manifold has a 25% compression load deflection in a range of about 1.05-1.75 pounds per square inch and a 65% compression load deflection in a range of about 1.29-2.15 pounds per square inch.

16. The fluid bridge of any of claims 1-15, wherein the open-cell foam of the support manifold comprises felted foam with a firmness factor of 2-5.

17. The fluid bridge of any of claims 1-15, wherein the open-cell foam of the support manifold comprises felted foam with a firmness factor of 3-5.

18. The fluid bridge of any of claims 1-17, wherein the support manifold has a length of at least about 650 mm.

19. The fluid bridge of any of claims 1-18, wherein the bridge has a width of about 40 mm.

20. The fluid bridge of any of claims 1-19, wherein the bridge has a length at least 15 times greater than the width.

21. The fluid bridge of any of claims 1-20, wherein the support manifold is configured so that a negative-pressure drop across the length of the fluid bridge when in substantially vertical orientation is no more than about 45 mmHg.

22. The fluid bridge of any of claims 1-20, wherein the support manifold is configured so that a negative-pressure drop across the length of the fluid bridge when in substantially vertical orientation is no more than about 50 mmHg.

23. The fluid bridge of any of claims 1-20, wherein the support manifold is configured so that a negative-pressure drop across the length of the fluid bridge when in substantially vertical orientation and under compression is no more than about 50 mmHg.

24. The fluid bridge of any of claims 1-20, wherein the fluid bridge is configured so that a negative-pressure drop across a length of the bridge is less than 60 mmHg when the bridge is in a vertical orientation.

25. The fluid bridge of any of claims 1-20, wherein the fluid bridge is configured so that a negative-pressure drop across a length of the bridge is less than 75 mmHg when the bridge is in a vertical orientation and under compression.

26. The fluid bridge of any of claims 2-25, wherein the fluid bridge is configured to maintain, when used in a vertical orientation, at least 75 mmHg negative pressure at the second aperture when 125 mmHg negative pressure is applied to the first aperture.

27. The fluid bridge of any of claims 2-25, wherein the fluid bridge is configured to maintain, when used in a vertical orientation and under compression, at least 75 mmHg negative pressure at the second aperture when 125 mmHg negative pressure is applied to the first aperture.

28. The fluid bridge of any of claims 2-27, wherein:
the envelope comprises a first film layer forming the first surface of the envelope and a second film layer forming the second surface of the envelope; and
the first film layer and the second film layer are coupled at a periphery to form the envelope.

29. The fluid bridge of any of claims 1-28, further comprising a wicking layer adjacent to the second surface, opposite the support manifold.

30. The fluid bridge of any of claims 1-29, wherein the envelope comprises a perforation providing controlled flow of less than about 5 cc/min located in proximity to the second end.

31. The fluid bridge of any of claims 1-30, further comprising a hydrophilic layer adjacent to the support manifold within the envelope.

32. A fluid bridge comprising:
a manifold comprising foam having open cells and a thickness of at least 5 mm, wherein the foam of the manifold comprises felted foam with a felted firmness factor of about 2-5; and
an envelope encompassing the manifold, wherein the manifold supports the envelope.

33. The fluid bridge of claim 32, wherein the foam of the manifold has a felted foam firmness factor of 3-5.

34. The fluid bridge of any of claims 32-33, wherein the foam of the manifold has pre-felted foam characteristics of 40-50 pores per inch on average and density of 1.3-1.6 lb/ft³.

35. The fluid bridge of any of claims 32-34, wherein the foam of the manifold has pre-felted average pore sizes of 400-600 micron.

36. The fluid bridge of any of claims 32-35, wherein the foam of the manifold has pre-felted thickness of 10-35 mm.

37. The fluid bridge of any of claims 32-35, wherein the foam of the manifold has pre-felted thickness of 10-25 mm.

38. The fluid bridge of any of claims 32-35, wherein the foam of the manifold has pre-felted thickness of 10-20 mm.

39. The fluid bridge of any of claims 32-35, wherein the foam of the manifold has pre-felted thickness of 15-20 mm.

40. The fluid bridge of any of claims 32-39, wherein:
the envelope comprises a first surface and a second surface;
the bridge comprises a first end and a second end; and
the envelope further comprises a first aperture located on the first surface in proximity to the first end and a second aperture located on the second surface in proximity to the second end.

41. The fluid bridge of any of claims 32-40, wherein the foam of the manifold is hydrophobic.

42. The fluid bridge of any of claims 32-41, wherein the foam of the manifold comprises polyurethane foam.

43. The fluid bridge of any of claims 32-42, wherein the foam of the manifold has a thickness of about 5-7 mm.

44. The fluid bridge of any of claims 40-43, wherein:
the envelope comprises a first film layer forming the first surface of the envelope and a second film layer forming the second surface of the envelope; and
the first film layer and the second film layer are coupled at a periphery to form the envelope.

45. The fluid bridge of any of claims 40-44, further comprising a wicking layer adjacent to the second surface, opposite the support manifold.

46. The fluid bridge of any of claims 32-45, wherein the envelope comprises a perforation providing calibrated flow of less than about 5 cc/min located in proximity to the second end.

47. A system for treating a tissue site with negative pressure, comprising:
a fluid bridge comprising a first end, a second end, and a support manifold having a free volume in a range of about 18% to about 45%; and
a negative-pressure source in fluid communication with the support manifold at the first end of the fluid bridge;
wherein the second end is configured to be in proximity to and in fluid communication with the tissue site.

48. The system of claim 47, further comprising a compression garment configured to cover at least the second end of the fluid bridge.

49. The system of claim 48, wherein the compression garment is configured to apply about 20-60 mmHg compression.

50. The system of claims 48, wherein the compression garment is configured to apply about 40-60 mmHg compression.

51. The system of any of claims 47-50, wherein the fluid bridge is configured so that a negative-pressure drop between the first end and the second end of the fluid bridge is no more than about 50 mmHg.

52. The system of any of claims 47-50, wherein the fluid bridge is configured so that a negative-pressure drop between the first end and the second end of the fluid bridge is less than 60 mmHg.

53. The system of any of claims 47-50, wherein the fluid bridge is configured so that a negative-pressure drop between the first end and the second end of the fluid bridge is less than 75 mmHg.

54. The system of any of claims 47-53, wherein the bridge is configured to maintain at least 75 mmHg negative pressure at the second end when 125 mmHg negative pressure is applied to the first end.

55. The system of any of claims 47-54, wherein the fluid bridge comprises any of claims 1-46.

56. A method of applying negative pressure to a tissue site, comprising:
providing a fluid bridge having a support manifold with a first end and a second end;
disposing the fluid bridge with the second end in proximity to the tissue site;
providing a compression garment;
disposing the compression garment to provide compression to the tissue site, wherein the second end is disposed under the compression garment; and
applying negative pressure to the first end;
wherein the second end maintains at least 75 mmHg negative pressure to the tissue site.

57. The method of claim 56, wherein a negative-pressure drop between the first end and the second end is no more than 50 mmHg.

58. The method of any of claims 56-57, wherein disposing the fluid bridge comprises orienting the fluid bridge substantially vertically.

59. The method of any of claims 56-58, wherein disposing the compression garment comprises configuring the compression garment to apply about 20-60 mmHg compression.

60. The method of any of claims 56-58, wherein disposing the compression garment comprises configuring the compression garment to apply about 40-60 mmHg compression.

61. The method of any of claims 56-60, wherein applying negative pressure comprises applying negative pressure of about 125 mmHg to the first end.

62. The method of any of claims 56-60, wherein the negative pressure applied to the first end is from about 125 to about 150 mmHg.

63. The method of any of claims 56-60, wherein the negative pressure applied to the first end is no less than 125 mmHg.

64. The method of any of claims 56-63, wherein applying negative pressure occurs continuously for at least 60 hours.

65. The method of any of claims 56-64, relating to or using the fluid bridge of claims 1-46.

66. A method of forming a fluid bridge, comprising:
providing an open-cell foam having a thickness of at least 5 mm, a density in a range of about 2.6-8.0 lb/ft³, and about 80-250 pores per inch on average; and
encasing the open-cell foam in an envelope, wherein the open-cell foam supports the envelope to maintain a fluid pathway.

67. The method of claim 66, wherein the open-cell foam has an average pore size ranging from 80-300 micron.

68. The method of any of claims 66-67, wherein the open-cell foam is hydrophobic.

69. The method of any of claims 66-68, wherein the open-cell foam comprises polyurethane foam.

70. The method of any of claims 66-69, wherein the open-cell foam has a thickness of about 5-7 mm.

71. The method of any of claims 66-70, wherein the open-cell foam has a density of about 3.9-4.8 lb/ft³.

72. The method of any of claims 66-71, wherein the open-cell foam has an average pore size that ranges from 133-200 micron.

73. The method of any of claims 66-72, wherein the open-cell foam has 120-150 pores per inch on average.

74. The method of any of claims 66-73, wherein the open-cell foam has 120-135 pores per inch on average.

75. The method of any of claims 66-74, wherein:
the envelope comprises a first surface and a second surface;
the bridge comprises a first end and a second end; and
the method further comprises forming a first aperture located on the first surface in proximity to the first end and a second aperture located on the second surface in proximity to the second end.

76. The method of any of claims 66-75, wherein providing an open-cell foam comprises:
providing an open-cell foam blank having thickness of at least 10 mm, 40-50 pores per inch on average, and density of 1.3-1.6 lb/ft³; and
felting the open-cell foam blank.

77. The method of claim 76, wherein felting the open-cell foam blank comprises felting the open-cell foam blank to 2-5 firmness.

78. The method of claim 76, wherein felting the open-cell foam blank comprises felting the open-cell foam blank to 3-5 firmness.

79. The method of claim 76, wherein felting the open-cell foam blank comprises felting the open-cell foam blank to firmness 3.

80. The method of any of claims 76-79, wherein the open-cell foam blank has average pore sizes of 400-600 micron.

81. The method of any of claims 76-80, wherein the open-cell foam blank has thickness of 10-35 mm.

82. The method of any of claims 75-81, further comprising attaching a wicking layer adjacent to the second surface, opposite the open-cell foam.

83. The method of any of claims 66-82, further comprising forming a perforation configured to provide calibrated flow into the envelope of less than about 5 cc/min located in proximity to the second end.

84. The method of any of claims 66-83, further comprising:
providing a hydrophilic foam layer; and
disposing the hydrophilic foam layer in proximity to the open-cell foam within the envelope.

85. The method of claim 84, wherein:
the first surface of the envelope comprises an evaporative layer; and
the hydrophilic foam layer is disposed between the open-cell foam and the first surface.

86. The method of any of claims 66-85, wherein the open-cell foam has a free volume in a range of about 18% to about 45%.

87. The method of any of claims 66-86, relating to the fluid bridge of claims 1-46.

88. The systems, apparatuses, and methods substantially as described herein.
